(19) United States
(12) Patent Application Publication
(10) Pub. No.: US 2012/0149984 A1
(43) Pub. Date: Jun. 14, 2012

(54) Heat-and Fire-Resistant Container for Holding Endoscope During Surgical Operation

(76) Inventor: Brian Kip Reilly, Chevy Chase, MD (US)

(21) Appl. No.: 13/008,003

(22) Filed: Jan. 17, 2011

Related U.S. Application Data
(60) Provisional application No. 61/336,190, filed on Jan. 19, 2010.

(51) Int. Cl. A61B 1/00

(52) U.S. Cl. 600/121

(57) ABSTRACT

The invention, referred to commercially as the “Endosleeve,” is a heat- and fire-resistant holster, holder or sleeve to secure and protect an endoscope during a medical or surgical operation. The Endosleeve consists of several components: a clamp or other mechanism that attaches the Endosleeve to the operating table; flexible tubing, which connects the clamp to the actual holster in which the endoscope is placed and which allows the physician to move the Endosleeve as necessary; and a holster that is fire and heat resistant, protecting the physician and patient from operating room fires. There is currently no such safety device to safely hold an endoscope during a medical or surgical procedure.

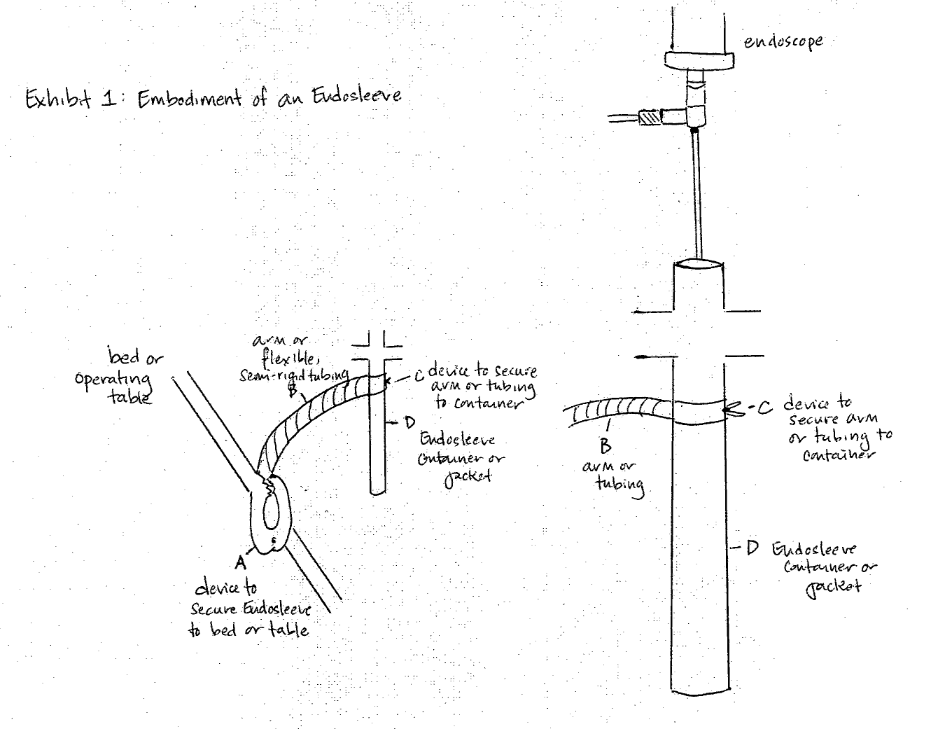

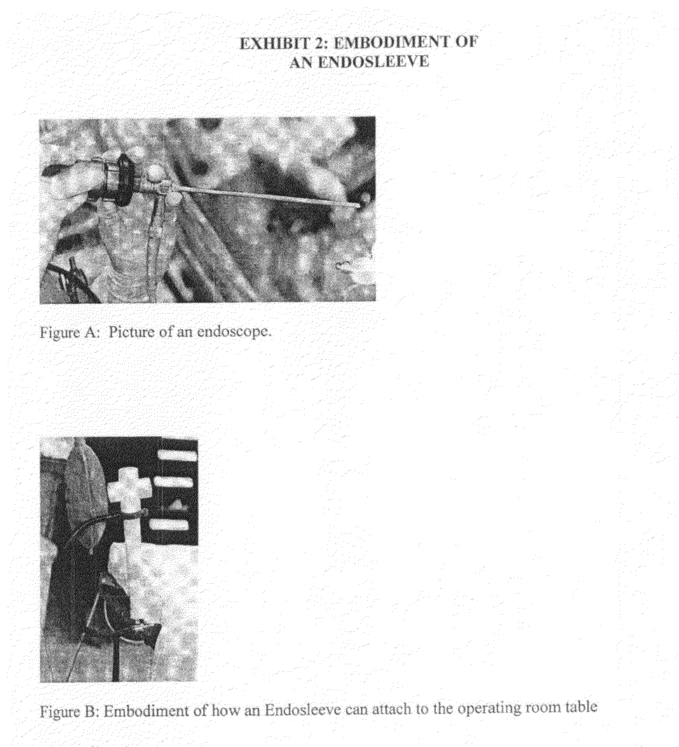

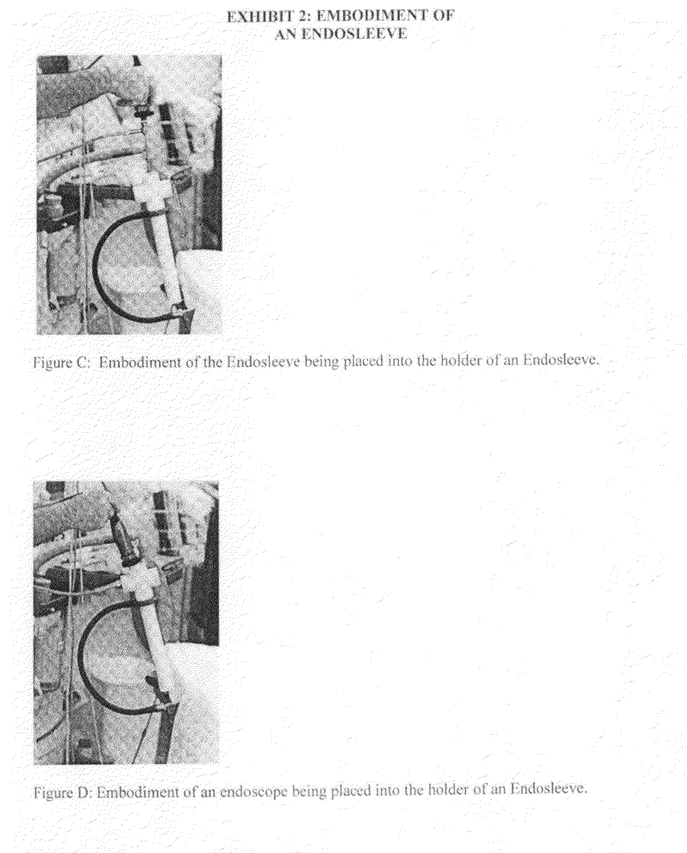

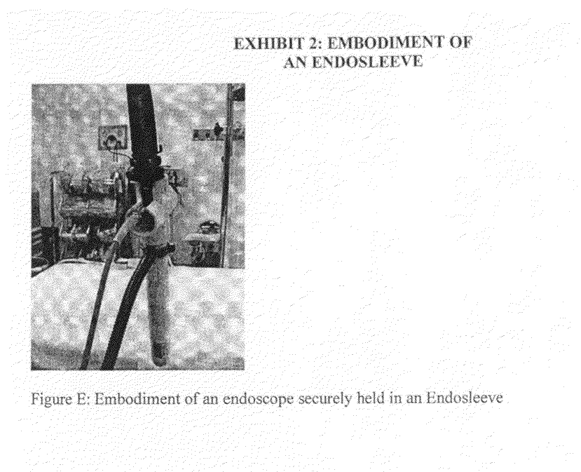

### CROSS REFERENCE TO RELATED APPLICATIONS

The following is inventor's provisional patent application, which relates to the instant patent application:

Application No.: 61/336,190

Application Filing Date: Jan 19, 2010

Relationship of the applications: The provisional patent application involves the same device for which inventor is seeking a nonprovisional patent.

### STATEMENT REGARDING FEDERALLY SPONSORED RESEARCH OR DEVELOPMENT

The invention was not made with the assistance of and does not involve any federally sponsored research or development.

### BACKGROUND OF THE INVENTION

1. Field to Which Invention Pertains

The invention pertains to an endoscope, a light carrier telescope used in surgery. The most relevant U.S. patent classification is Class 600 (Surgery)/101 (Endoscope).

2. Problem That the Invention is Designed to Solve

The purpose of this invention, the “Endosleeve,” is to act as a holster, holder or sleeve (hereinafter “holster”) to secure and protect a surgical light carrier telescope (also commonly known as an endoscope) during a medical or surgical operation. Surgical telescopes/endoscopes are rigid light carrying metal tubes which contain optical lenses used in multiple medical procedures, including abdominal procedures; complex paranasal sinus and skull base surgery; orthopedic surgeries involving the shoulder, knee or the hip; and urologlogical and gynecological surgeries.

At present, there is no device designed specifically to safely hold an endoscope during surgery. Instead, the surgeon has to either lay the endoscope down on the surgical drapes over the patient or have an assistant (e.g. nurse) hold it over the patient once the surgeon is no longer holding or using the endoscope (e.g., if the surgeon wants to intermittently use another instrument). By laying the endoscope down on the patient, or at the patient's bedside, the surgeon runs the risk of a fire in the operating room. Alternatively, having an assistant hold the endoscope while the surgeon is not using it decreases the efficiency of the procedure and increases the time in which the patient must remain under anesthesia.

### BRIEF SUMMARY OF INVENTION

The “Endosleeve” is a fire-resistant holster, which allows the surgeon to safely store an endoscope during surgery. The Endosleeve is attached to the patient's operating table but has flexible tubing allowing the surgeon to maneuver the holster as necessary. In this way, the Endosleeve will prevent operating room fires. Each year, there are approximately 100 surgical fires documented, resulting in up to 20 serious injuries and one or two patient deaths annually. “Preventing Surgical Fires,” Sentinel Event Alert 29 (Jun. 24, 2003), at http://www.jcaho.org/about us/news letters/sentinel event alert/print/sea_{—}29.htm (accessed Jan. 9, 2010).

In addition, the Endosleeve will improve the efficiency for the operating surgeon. If the nurse or physician's assistant is required to hold the telescope while the surgeon uses different instruments, the assistant is less efficient and does not have a free hand to organize, select and pass the additional instrument to the surgeon. This adds clutter to an already cramped surgical environment.

### BRIEF DESCRIPTION OF THE SEVERAL VIEWS OF THE DRAWING

Exemplary embodiments of the present disclosure are illustrated in the accompanying drawing. The drawing is not to scale but is rather is meant to illustrate the principles of the present disclosure.

Exhibit 1: This is a diagram of an embodiment of an Endosleeve. It depicts each of the component parts:

A) “A” depicts a device that properly secures the “Endosleeve” to the patient's bed or other operating room surface. This device may be a clamp, clasp, lock, screw, joint, an attachment or other similar device. The device also may have a variety of shapes, forms and sizes.

B) “B” illustrates the flexible, semi-rigid tubing that would be used for the Endosleeve. This tubing will allow the surgeon to adjust the height and angle of the Endosleeve. Alternatively, the Endosleeve can be made with any arm that allows for the adjustment of the position of the sleeve.

C) “C” depicts a device that secures the tubing to the container used to hold the endoscope, i.e., the “Endosleeve jacket.” This device may be a clamp, screw, bracket or other fastener, or other similar device. The tubing may also be directly fastened or fused to the container.

D) “D” illustrates the “Endosleeve jacket.” This jacket will be fire and heat resistant to ensure that when the surgeon puts the endoscope down, it will not rest on the patient or come in contact with the drapes. The jacket may have a variety of shapes and sizes.

Exhibit 2: These are photographs of an embodiment of a prototype of an Endosleeve to demonstrate how the Endosleeve works in practice.

A) Figure A depicts an endoscope.

B) Figure B is an embodiment of how an Endosleeve attaches to the operating table.

C) Figure C is an embodiment of an endoscope being placed into an Endosleeve.

D) Figure D is an embodiment of the endoscope being placed into an Endosleeve.

E) Figure E also is an embodiment of the endoscope being securely held by the Endosleeve.

### DETAILED DESCRIPTION OF THE INVENTION

One embodiment of the present disclosure, the Endosleeve, can be made by following the embodiment drawn in Exhibit 1 and depicted in the photographs in as Exhibit 2.

The Endosleeve requires a simple clamp for proper attachment to the patient's bed or other operating room surface. For this purpose, any type of secure attachment will suffice, including but not limited to a clamp, clasp, lock, screw, bracket, joint or other attachment. Persons of ordinary skill in the art would understand that the secure attachment may have a variety of shapes, forms and sizes. (Exhibit 1, “A”). Flexible tubing is then attached or fused to the clamp or other secure device. (Exhibit 1, “B”). Ideally, this tubing must be flexible enough to allow the surgeon to bend and move the non-flammable container (the “Endosleeve jacket”—Exhibit 1, “D”) in which the rigid telescope will be placed but also rigid enough to be able to withstand the weight of the endoscope and to be able to “freeze” the Endosleeve and endoscope in the precise location selected by the operating surgeon. The tubing may be hollow or solid, and can be made from any material that will satisfy the criteria described above. Alternatively, the Endosleeve can be made with any arm that allows for the adjustment of the position of the sleeve.

The tubing then clamps on to the “Endosleeve jacket” (Exhibit 1, “C”—depicting the clamp; Exhibit 1, “D”—depicting the “jacket”). The tubing may be attached to the jacket using a number of devices, including but not limited to a clamp, clasp, screw, joint, bracket, lock or other secure device. The tubing may also be directly fused to the jacket. In Exhibits 1 and 2, the “jacket” is shown as a heat and fire resistant plastic tube in a cross-shape, which opens on one side to accommodate the wiring of a telescope. Depending on the size and shape of the scopes, the size and shape of the container may vary. Persons of ordinary skill in the art would understand that the Endosleeve jacket may have a variety of shapes and sizes. The material may also vary, but will be a non-flammable substance, which has the ability to securely hold the endoscope. As Exhibits 1 and 2 demonstrate, the endoscope can be placed directly in the container for safe keeping while the endoscope is not in use.

All materials used in the production of the Endosleeve will be heat and fire resistant, and will have the ability to be cleaned and sterilized in accordance with standard operating room procedures.

1. A medical device comprising:
A container or holder suitable for holding an endoscope;
An arm attached to said container or holder that permits the position of the container or holder to be adjusted;
A device that secures said arm to a surface.

2. The medical device of claim 1 in which the container or holder is fire and heat resistant.

3. The medical device of claim 1 in which the size and shape of the container or holder is tailored to match the specific endoscope in use.

4. The medical device of claim 1 in which the arm is comprised of flexible, semi-rigid tubing.

5. The medical device of claim 1 in which the device that secures the arm to a surface is a clamp.

6. The medical device of claim 1 in which the device that secures the arm to a surface is a clasp.

7. The medical device of claim 1 in which the device that secures the arm to a surface is a bracket.

8. The medical device of claim 1 in which the device that secures the arm to a surface is a screw.

9. The medical device of claim 1 in which the device that secures the arm to a surface is a joint.

10. The medical device of claim 1 in which the device that secures the arm to a surface is a lock.

11. The medical device of claim 1 in which a device is used to secure the arm to the container or holder.

12. The medical device of claim 11 in which the device used to secure the arm to the container or holder is a clamp.

13. The medical device of claim 11 in which the device used to secure the arm to the container or holder is a clasp.

14. The medical device of claim 11 in which the device used to secure the arm to the container or holder is a bracket.

15. The medical device of claim 11 in which the device used to secure the arm to the container or holder is a screw.

16. The medical device of claim 11 in which the device used to secure the arm to the container or holder is a joint.

17. The medical device of claim 11 in which the device used to secure the arm to the container or holder is a lock.
